(19) United States
(12) Patent Application Publication
(10) Pub. No.: US 2024/0374625 A1
(43) Pub. Date: Nov. 14, 2024

(54) TREATMENT OF SCHIZOPHRENIA WITH PDK1 INHIBITION BY COMBINING PDK1 INHIBITORS WITH LIPID REPLACEMENT THERAPY

(71) Applicant: Adonis Sfera, Redlands, CA (US)

(72) Inventor: Adonis Sfera, Redlands, CA (US)

(21) Appl. No.: 18/633,444

(22) Filed: Apr. 11, 2024

Related U.S. Application Data
(60) Provisional application No. 63/496,685, filed on Apr. 17, 2023.

(51) Int. Cl. A61K 31/7032; A61K 31/366; A61K 31/415; A61K 31/498; A61K 31/501; A61K 31/519; A61K 31/5377; A61K 31/541; A61K 31/5415; A61K 31/685; A61K 45/06

(52) U.S. Cl. CPC A61K 31/7032; A61K 31/366; A61K 31/415; A61K 31/498; A61K 31/501; A61K 31/519; A61K 31/5377; A61K 31/541; A61K 31/5415; A61K 31/685; A61K 45/06

(57) ABSTRACT

Schizophrenia is characterized by dysfunction of neuronal membrane bilayers that disrupt normal neurotransmission. This can also be exacerbated by long term use of current antipsychotic drugs and dopamine depletion. Small molecule PI3K/PDK1 inhibitors may restore neuronal membrane integrity are therefore of potential interest as anti-psychotic or anti-schizophrenic drugs. Similarly, Lipid Replacement Therapy, with at least two components selected from phosphatidylcholine; phosphatidyl-inositol; phosphatidylethanolamine; phosphatidic acid; and digalactosyl-diacylglycerol can disrupt the membrane dysfunction and are of value in treating psychosis such as schizophrenia. may be more effective treatments for long term use. A number of small molecule PI3K/PDK1 inhibitors and Lipid Replacement Therapy formulations are disclosed. Also disclosed are combinations of these modalities and pharmaceutical compositions.

### FIELD OF THE INVENTION

This invention relates to the treatment of psychosis such as schizophrenia by the inhibition of 3-phosphoinositide-dependent protein kinase 1 (PDK1) with small molecule PDK1 inhibitors or lipid replacement therapy, or a combination of small molecule PDK1 inhibitors with lipid replacement therapy.

### BACKGROUND

Schizophrenia is a severe neuropsychiatric illness, affecting approximately 1% of the world population, that despite current treatments, progresses gradually toward disability and cognitive deficit. This emphasizes an unmet need, the development of etiopathogenetic therapies that could stop the evolution of this disorder [1]. Recent studies, including the ENIGMA Schizophrenia Working Group, linked this illness to premature brain aging, a phenotype previously associated with dysfunctional cell membranes. [2-5] Cellular senescence may also play a role. Senescent neurons, like senescent somatic cells, are characterized by oxidized membrane lipids that disrupt neurotransmission, leading to pathology, including schizophrenia. [6-9] In this disclosure, the term “schizophrenia” is used throughout, but other severe psychiatric disorders generally termed psychoses are within the scope of this term as used herein. These disorders fall in ICD-10 classes F20-F29.

The available antipsychotic drugs excel at eliminating the positive symptoms in acute psychosis and they will likely remain the standard of care for the foreseeable future. However, exposure to these agents and chronic dopamine (DA) depletion was demonstrated to disrupt synaptic plasticity, contributing to impaired new information learning.[10] When superimposed on late-life dementia, dysfunctional neuroplasticity leads to more pronounced cognitive deficits, consistent with a distinct etiopathogenetic entity. Indeed, under physiological circumstances, healthy aging was associated with increased synthesis of striatal DA, probably to compensate for the age-related downregulation of DA receptors. Loss of synaptic plasticity, a modifiable risk factor, may be reversed with neurolipidomic therapies and/or DA reuptake inhibitors (DRIs).

Akt-induced senescence (AIS) refers to the premature aging of cells, including the neurons, mediated by PI3K-PKB/Akt (phosphoinositide-3-kinase-protein kinase B (PKB) also known as Akt). This cell membrane pathway, implicated in schizophrenia,[11] is activated by phosphoinositide-dependent kinase 1 (PDK1), a master regulator of phosphorylation. [12] The aging process has been associated with Akt phosphorylation and activation, suggesting that, unlike previously believed, schizophrenia-mediated premature brain aging also activates Akt. [13,14] Several antipsychotic drugs, including clozapine and risperidone, are Akt inhibitors (which suppress Akt phosphorylation), while dopamine (DA) is an Akt activator (which promote phosphorylation of this biomolecule). [13, 15] In contrast, haloperidol upregulates the Akt pathway, likely by altering the biophysical properties of plasma membrane [16] (FIG. 1). Table 1 summarizes the similarities and differences between the role of Akt activators and inhibitors in psychotic disorders. It is noteworthy that the second-generation antipsychotic drugs inhibit Akt, while many first-generation therapeutics intercalate in the cell membrane bilayer, activating Akt (FIG. 1).

TABLE 1
Akt activators and inhibitors relevant for schizophrenia

| Akt activators | Akt inhibitors | References |
|---|---|---|
| Dopamine | Clozapine | [13], [15] |
| Haloperidol | Risperidone | [13], [16] |
| Cellular senescence | PDK1 inhibitors | [9], [12], [17] |
| Neurotoxic microglia | LRT | [18], [19], [20] |

### Rationale for Targeting PI3K/Akt Signaling Pathway

Several lines of research suggesting that targeting the PI3K/Akt signaling pathway may be useful to treat psychotic disorders are described below.

Senescent neurons, as evidenced by studies such as those by Katsel et al. [21] and Okazaki et al. [22] display aberrant indices of cell cycle activity, with particular relevance to the dysregulation of cyclin E/Cdk2 signaling pathways in schizophrenia. Additionally, research by Bhaskar et al. [23] underscores the pivotal role of the PI3K-Akt-mTOR pathway in mediating the reactivation of the cell cycle in response to pathological stimuli, such as amyloid-beta oligomers, implicating this pathway in both neurodegenerative disorders and schizophrenia. Furthermore, insights from Nandakumar et al. [24] shed light on the broader implications of cell cycle re-entry in the nervous system, highlighting its association with neurodegeneration and underscoring its relevance to understanding the pathophysiology of various neurological disorders.

Likewise, malignant cells exploit the PI3K/Akt pathway, as elucidated by Gao et al. [25], to instigate aberrant activation of the cell cycle machinery. This dysregulated signaling cascade engenders the overexpression of G1 cyclins, particularly cyclin E/Cdk2. This intricate molecular hijacking fosters unchecked cell cycle progression, contributing to the unrestrained proliferation characteristic of cancerous states. Furthermore, Liang and Slingerland [26] underscore the multifaceted involvement of the PI3K/PKB (Akt) pathway in orchestrating various facets of cell cycle progression, further accentuating the pivotal role of this signaling axis in driving malignant cellular behavior. This suggests a possible role for PI3K/Akt upregulation in psychotic disorders, and suggests that inhibitors of PI3K/Akt, including those developed for cancer therapy may be value in the treatment of neuoropsychiatric disorders including schizophrenia.

Inhibition of 3-phosphoinositide-dependent protein kinase 1 (PDK1), part of the PI3K/Akt cascade,[11] has emerged as a promising therapeutic strategy with implications spanning diverse pathological conditions. Notably, research by An et al. [27] illuminates the pivotal role of PDK1 inhibition in reversing cellular senescence, particularly evident in human dermal fibroblasts. This finding underscores the potential of PDK1 inhibition as a means to counteract senescent hallmarks, offering a glimmer of hope in combating age-related degenerative disorders such as Alzheimer's disease (AD). Furthermore, the therapeutic relevance extends beyond aging-related afflictions, as elucidated by Yang et al. [28], who highlights the efficacy of reducing PDK1/Akt activity in ameliorating AD pathology. This suggests a broader spectrum of beneficial effects, hinting at the potential utility of PDK1 inhibition in mitigating other neurodevelopmental disorders such as schizophrenia, thereby offering novel avenues for therapeutic intervention in complex neurological conditions.

Inhibition of Akt/PDK1 signaling is also a promising avenue for neuroprotection and facilitation of axonal regeneration, as delineated in recent studies. Yang et al. [28] This underscores the therapeutic potential of reducing PDK1/Akt activity, particularly evident in the context of Alzheimer's disease (AD) treatment. This inhibition not only holds promise for mitigating AD pathology but also signifies broader neuroprotective effects, potentially fostering neuronal survival and functional recovery.

Moreover, insights from Kim et al. [29] elucidate the role of PDK1 as a negative regulator of axon regeneration. By targeting PDK1, it may be possible to unleash the intrinsic regenerative capacity of neurons, facilitating axonal regrowth and neural circuit remodeling. These findings collectively highlight the dual benefits of Akt/PDK1 inhibition in safeguarding neurons from degenerative insults while simultaneously promoting axonal regeneration, thus offering a multifaceted therapeutic approach for neurological disorders characterized by neuronal damage and impaired connectivity.

Activation of the PI3K/PDK1/Akt pathway also precipitates a cascade of events culminating in the activation of neurotoxic microglia, posing a substantial threat to neuronal and synaptic integrity, as evidenced by studies in schizophrenia and neurodegenerative disorders. Rai et al. [19] elucidate the pivotal role of PI3K/Akt signaling in the pathogenesis of neurodegenerative disorders, shedding light on its contribution to neurotoxicity mediated by microglia. This aberrant activation of microglia, driven by dysregulated PI3K/PDK1/Akt signaling, manifests as the elimination of healthy neurons and synapses, exacerbating neuronal dysfunction and cognitive decline observed in these disorders.

### Inhibition of Microglia

Furthermore, insights from Laskaris et al. [30] corroborate the deleterious impact of microglial activation on brain integrity, particularly in schizophrenia. Their findings highlight the progressive nature of microglial activation and its association with structural and functional alterations in the schizophrenic brain. This convergence of evidence underscores the detrimental consequences of PI3K/PDK1/Akt-mediated neuroinflammation, implicating it as a key player in the pathophysiology of both schizophrenia and other neurodegenerative diseases.

In contrast, PDK1 inhibitors and PI3K blockers, exemplified by ZSTK474,[31] exert a potent moderating influence on microglial activation, thereby mitigating the cascade of neuroinflammatory responses and safeguarding against synaptic and neuronal loss, as delineated in recent investigations. Cianciulli et al. [20] elucidate the pivotal role of PI3K modulation in regulating microglial-mediated neuroinflammation. Through inhibition of PDK1 and PI3K, these compounds intervene in the activation of microglia, tempering their inflammatory phenotype and attenuating the detrimental consequences on neuronal and synaptic integrity.

Moreover, Wang et al. [32] provide further insights into the therapeutic potential of PI3K inhibition, specifically with ZSTK474, a blood brain barrier (BBB) penetrable PI3K inhibitor,[33] in the context of cerebral ischemia/reperfusion injury. Their findings underscore the ability of ZSTK474 to induce a shift in microglial/macrophage phenotype towards a less inflammatory state, thereby curbing the inflammatory response implicated in neuronal damage. This convergence of evidence underscores the promise of PDK1 inhibitors and PI3K blockers as potential therapeutic agents for mitigating neuroinflammation-associated synaptic and neuronal loss, offering new avenues for intervention in neurodegenerative and ischemic disorders.

The discovery of PDK1 inhibitory properties associated with phenothiazines illuminates a striking convergence between antipsychotic drugs and PDK1 in their action mechanisms, as disclosed by Han [34] Phenothiazines, a class of compounds primarily utilized in the treatment of psychiatric disorders, have long been recognized for their multifaceted pharmacological effects. Han's insights underscore a previously unrecognized facet of phenothiazine action, implicating PDK1 inhibition as a potential mechanism underlying their therapeutic efficacy.

This revelation holds profound implications for our understanding of both psychiatric pharmacotherapy and intracellular signaling pathways. By elucidating the shared action mechanism between antipsychotic drugs and PDK1, Han's findings offer a new perspective on the molecular underpinnings of psychiatric disorders and hint at novel therapeutic targets. Moreover, this discovery opens avenues for the repurposing of phenothiazines or the development of new PDK1-targeted therapies, potentially bridging the gap between psychiatric and molecular pharmacology. Overall, the identification of PDK1 inhibition as a common feature of phenothiazines underscores the intricate interplay between pharmacology and cellular signaling pathways, paving the way for innovative approaches in both fields.

### Cell Membrane Lipidome

The currently available antipsychotic drugs are effective for the treatment of acute psychotic disorders, however, their efficacy in chronic psychosis (i.e., long-term use, over a period of many months or years) is much less dramatic and may even predispose to neurocognitive disorders by the lipid peroxidation of cell membranes. Dysfunctional cell membranes may lead to increased permeability of the gut barrier and BBB, enabling microbial translocation from the GI tract into the systemic circulation, eventually reaching the brain.

Neurolipidomics is a new systems biology field which attempts to elucidate the physiological and pathological role of the Central Nervous System (CNS) lipids. [34] Cells, including neurons, are surrounded by biological membranes, comprised of proteins and phospholipid bilayers, that separate the intracellular from the extracellular compartment, facilitating selective transport of nutrients and signaling molecules across the membranes.

Defective lipids in neuronal membranes were shown to alter both the cell integrity and neurotransmission, likely contributing to pathology, including schizophrenia. [35-37] For example, oxidized sphingolipids and polyunsaturated fatty acids (PUFA's) were found in the neuronal membranes of schizophrenic patients, suggesting that replacing these molecules with natural, dietary glycerophopsholipids may comprise a useful antipsychotic treatment modality. [38], [39] By the same token, microglial phenotype has been demonstrated to depend on the lipid composition of its cell membranes, suggesting that neurotoxic microglia could be tamed via LRT. For example, dietary fatty acids, such as docosahexaenoic acid and other omega-3 fatty acids, were demonstrated to shift microglial phenotype from neurotoxic to neuroprotective, highlighting further the crucial role of cell membrane lipids. [40-42]

Lipid replacement therapy (LRT) is a technique that utilizes natural glycerophospholipids to substitute oxidized components of the plasma membranes lipid bilayer, restoring the physiological fluidity as well as neurotransmitter signaling. This approach, based on the oral supplementation with natural phospholipids and antioxidants, was demonstrated to halt the dissemination of cellular senescence to the neighboring, healthy cells, probably by inhibiting a senescence associated secretory program (SASP). [7, 43] Moreover, LRT was shown to supplant not only cell membranes but also the damaged mitochondrial inner and outer membranes with new and natural lipid species. Indeed, loss of lysophosphatidylethanolamine (LPE), phosphatidylglycerol (PG), and phosphatidylinositol (PI) in senescent mitochondria was shown to cause organelle demise; conversely, replacement with healthy lipids promotes mitochondrial thriving.

### SUMMARY OF THE INVENTION

This invention provides for the use of small-molecule PI3K/PDK1 inhibitors and/or Lipid Replacement Therapy to treat or prevent the onset of schizophrenia. The invention also provides for the use of Lipid Replacement Therapy to treat or prevent the onset of schizophrenia. This invention also provides for combinations of small-molecule PI3K/PDK1 inhibitors and Lipid Replacement Therapy to treat or prevent the onset of schizophrenia. Formulations and compositions are also disclosed.

### DESCRIPTION OF THE DRAWINGS

FIG. 1. Schematic of neuronal cell membrane bilayer showing the interplay of lipids and PI3K/PDK1/Akt modulators. Neuronal membranes are comprised of a lipid bilayer that anchors numerous neurotransmitter receptors. Dysfunction in the membrane bilayer can be caused by antipsychotic drugs or excessive iron trigger, causing peroxidation of the lipid bilayer, altering membrane permeability. Intracellular iron activates downstream SCZ-related kinases, including Akt and PI3K contributing to the pathogenesis of this disease. Psychotropic drugs, especially Phenothiazines, intercalate themselves into the lipid bilayer, changing the biophysical properties of plasma membranes, which, in turn, disrupts neurotransmission as altered intracellular responses. Lipid replacement therapy may reverse this process.

FIG. 2. LRT inhibits PDK1 as exogenous phosphatidylserine (PS) binds the PH domain of PDK1, inactivating the downstream kinases, protein kinase B (Akt), and glycogen synthase kinase 3 beta (GSK-3β). LRT and PDK1 inhibitors thereby act as natural antipsychotic agents. The point of contact between exogenously administered phospholipids and PDK1 is phosphatidylserine (PS), which binds PDK1 through its PH domain, blocking PS externalization and the initiation of cellular senescence. Moreover, PH-PS attachment inhibits PDK1 activation of downstream kinases, accounting for the antipsychotic actions of PDK1 inhibitors. The novel PDK1 inhibitor OSU 03012 readily crosses the BBB, enhancing the antipsychotic properties of LRT.

### DETAILED DESCRIPTION

This invention provides for the use of small-molecule PI3K/PDK1 inhibitors to treat or prevent the onset of schizophrenia. The invention also provides for the use of Lipid Replacement Therapy to treat or prevent the onset of schizophrenia. This invention also provides for combinations of small-molecule PI3K/PDK1 inhibitors and Lipid Replacement Therapy to treat or prevent the onset of schizophrenia. Formulations and compositions are also disclosed.

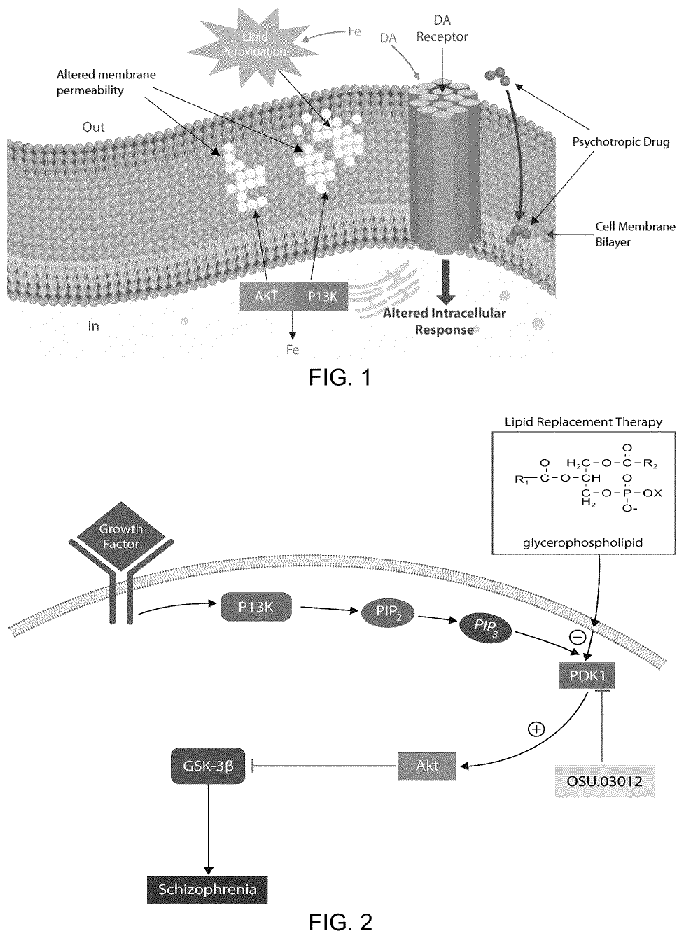

Schizophrenia causes premature brain aging by several measures. Prematurely aged brains in schizophrenia retain iron (FIG. 1), increasing the risk of lipid peroxidation and ferroptosis. Lipid peroxidation activates the Akt pathway, further increasing the brain iron levels. Moreover, dopamine (DA), functioning as a siderophore, can release iron, increasing the risk of ferroptotic neurona and neuronal death. Oxidized cell membranes disrupt neurotransmission by altering the biophysical properties of the lipid bilayer. Several first-generation psychotropic drugs, such as haloperidol and chlorpromazine, intercalate themselves into the lipid bilayer, altering the membrane properties and neurotransmitter, including DA, signaling. For this reason, haloperidol activates, while second-generation antipsychotic drugs deactivate Akt (Table 1).

Lipid Replacement Therapy (LRT) consists of oral supplementation of membrane glycerophospholipids to provide replacement molecules that are damaged by oxidation in several disorders, including schizophrenia. Given that the LRT component phosphatidylserine (PS), maintains PDK1 in an inactive conformation, this intervention may benefit patients with schizophrenia by inhibiting the downstream Akt and GSK-33 phosphorylation. Also, LRT may restore not only the physiological cell membrane function but also the integrity of biological barriers, such as gut barrier (GB) and blood-brain barrier (BBB). The addition of PI3K-PKB/Akt affects the same cascade and is expected to have the same effects.

Akt is a serine-threonine protein kinase that plays important roles in cell growth, proliferation and apoptosis. It is activated after binding to phosphatidylinositol phosphates (PIPs) with phosphate groups at positions 3,4 and 3,4,5 on the inositol ring.[44] PIPs are phospholipids composed of a glycerol backbone with fatty acids. Upon growth factor-receptor binding, PDK-1 is recruited to the cell membrane and activated by PI3K via PIP1 and PIP2 (FIG. 2). LRT, especially the glycerophospholipid phosphatidylserine (PS), inhibits PDK1, by maintaining it in inactive conformation. Downstream, Akt phosphorylate is inhibited, releasing the inhibitory break from GSK-33 (lack of inhibitory phosphorylation). Together, phosphorylation changes contribute to neuropathology, including schizophrenia. Some second-generation antipsychotic drugs upregulate GSK-33 (by increasing activating phosphorylation). OSU-03012, a small molecule PDK1 inhibitor, acts synergistically with LRT, inhibiting PDK1.

We surmise that combining LRT with inhibitors of phosphoinositide-dependent kinase 1 (PDK-1), such as kaempferol, would produce superior results in SCZ compared to LRT alone (FIG. 2). Indeed, PDK1 inhibitors reverse cellular senescence, a phenotype previously considered irreversible.[10] Lowered SASP together with the healthy exogenous lipids decrease the risk of peroxidation and ferroptosis. Interestingly, kaempferol is also an antagonist of AhR, a transcription factor and cellular senescence driver. On the other hand, PDK1 activation of protein kinase B (Akt) and glycogen synthase kinase 3 beta (GSK-3β) promotes SCZ pathogenesis. The point of contact between exogenously administered phospholipids and PDK1 is phosphatidylserine (PS), which binds PDK1 through its PH domain, blocking PS externalization and the initiation of cellular senescence. Moreover, PH-PS attachment inhibits PDK1 activation of downstream kinases, accounting for the antipsychotic actions of PDK1 inhibitors.[10]

This hypothesis is supported by the following findings:

PDK1 inhibitors and antipsychotic drugs share a common action mechanism: Akt inhibition. [45-46]

Cocaine, a drug associated with psychosis (due to increased brain DA) activates Akt, linking this molecule to schizophrenia. [47]

PDK1/Akt inhibition averts the development of AIS, likely alleviating schizophrenia-induced premature brain senescence.

PDK1 activation has been associated with AD-mediated psychosis, connecting AIS to schizophrenia. [48]

Several senolytic drugs including N-acetyl-cysteine (NAC) and alpha lipoic acid, possess antipsychotic properties, further linking psychosis to brain senescence. [49], [50]

Monoamine oxidase type B (MAO-B), a schizophrenia susceptibility gene, is inhibited by the cell membrane lipid, phosphatidylserine (PS), a component of LRT, highlighting an alternative antipsychotic mechanism of phospholipids. [51-52]

Flavonoids, natural compounds that have showed beneficial effects in schizophrenia, are Akt inhibitors, further linking this pathway to psychosis. [53-55]

Akt pathway inhibits the downstream GSK-3β, a molecule downregulated in schizophrenia, suggesting that Akt is overactive in this disorder and PDK1 inhibitors could restore its homeostasis. [56]PDK1 Inhibitors

A number of PDK1 inhibitors have been identified. For the most part, these compounds have been developed as anticancer drugs. The use of this class of drugs for psychiatric disorders including schizophrenia is novel. Several exemplary PDK1 inhibitors are discussed below. It is important that any PDK1 inhibitor be able to cross the blood brain barrier (BBB) to be effective as a treatment for psychiatric disorders.

OSU-03012 (Table 3) is a PDK1 inhibitor and celecoxib derivative with antiproliferative, antibacterial and antiviral properties [17, 57, 58] (FIG. 2) (Table 2). Antipsychotic drugs share these OSU-03012 properties, indicating a shared action mechanism [59-61] (Table 2).

TABLE 2
Common characteristics of antipsychotic drugs and OSU-03012

| OSU-03012 | Antipsychotic drugs | References |
|---|---|---|
| Lowers ER stress via PERK | Lower ER stress via PERK | [62] |
| Inhibits cathepsins | Inhibit cathepsins | [62], [63], [64] |
| Antineoplastic properties | Antineoplastic properties | [17], [60] |
| Antibacterial properties | Antibacterial properties | [58], [59] |
| Antiviral properties | Antiviral properties | [57], [61] |

OSU-03012 is a member of the class of pyrazoles, particularly N-[4-(pyrazol-1-yl)phenyl]glycinamide in which the pyrazole ring is substituted at positions 3 and 5 by trifluoromethyl and phenanthreneyl-2 groups respectively.

OSU-03012 (also known as AR-12) is an orally bioavailable, small-molecule with potential antineoplastic properties. [17] Devoid of any COX inhibiting activity, OSU-03012 binds to and inhibits PDK1. Subsequently, phosphorylation and activation of Akt is inhibited, which may also inhibit the PI3K/Akt signaling pathway. In addition, like lysosomotropic antipsychotic drugs, OSU-03012 inhibits cathepsins and activates the PKR-like endoplasmic reticulum kinase (PERK), which plays a key role in lowering endoplasmic reticulum (ER) stress as part of the unfolded protein response (UPR) [62-65] (Table 2). Activation and dysregulation of the PI3K/Akt signaling pathway is associated with both tumorigenesis and schizophrenia.

OSU-03012 phase I human studies found that at the dose of 800 mg twice daily, signs of PDK1 modulation were observed in concordance with the expected mechanism of action [18] (NCT00978523).

Other novel PI3K/Akt inhibitors and phenothiazine derivatives have been developed for cancer which prevent lipid peroxidation. [66], [67] Many of these compounds are devoid of extrapyramidal side effects and may be of value in schizophrenia therapy according to this invention. A number of PI3K/Akt inhibitors that

have been reported in the literature are listed in Table 3.

TABLE 3
Small molecule PI3K/PDK1 Inhibitors

| Structure | Name(s) | Ref. |
|---|---|---|
| 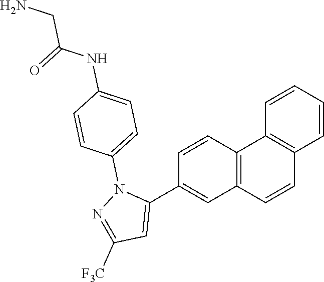 [SMILES: NCC(=O)Nc1ccc(-n2nc(C(F)(F)F)cc2-c2ccc3c(ccc4ccccc43)c2)cc1] | OSU-0312 AR-12 | [17] |
| 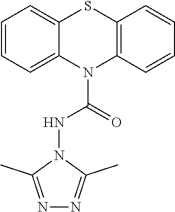 [SMILES: Cc1nnc(C)n1NC(=O)N1c2ccccc2Sc2ccccc21] | DT-PTC-Z; N-(3,5-Dimethyl- 4H-1,2,4-triazol-4-yl)-10H- phenothiazine-10- carboxamide | [66] |
| 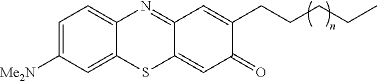 | MV derivatives | [67] |
| n = 4, 5, 6, 8 | | |
| 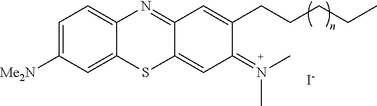 | MB derivatives | [67] |
| n = 4, 5, 6, 8 | | |
| 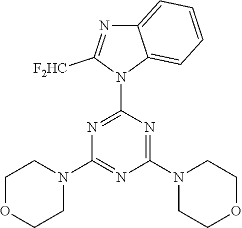 [SMILES: FC(F)c1nc2ccccc2n1-c1nc(N2CCOCC2)nc(N2CCOCC2)n1] | ZSTK474 | [31] |
| 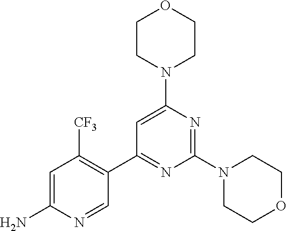 [SMILES: Nc1cc(C(F)(F)F)c(-c2cc(N3CCOCC3)nc(N3CCOCC3)n2)cn1] | BKM-120 (Buparlisib) | [31] |
| 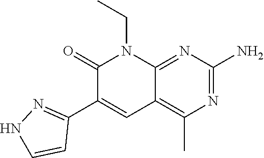 [SMILES: CCn1c(=O)c(-c2cc[nH]n2)cc2c(C)nc(N)nc21] | XL-765 (Voxtalisib) | [31], [68] |
| 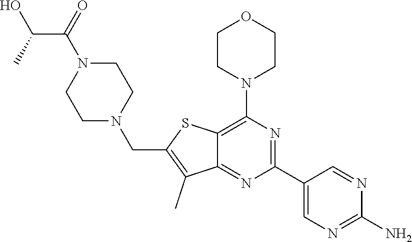 [SMILES: Cc1c(CN2CCN(C(=O)[C@H](C)O)CC2)sc2c(N3CCOCC3)nc(-c3cnc(N)nc3)nc12] | GDC-0980 (Apitolisib) | [31] |
| 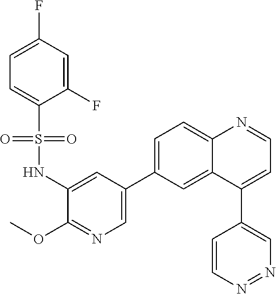 [SMILES: COc1ncc(-c2ccc3nccc(-c4ccnnc4)c3c2)cc1NS(=O)(=O)c1ccc(F)cc1F] | GSK-2126456 (Omipalisib) | [31] |
| 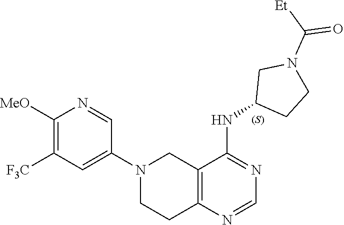 | Leniolisib JOENJA ® | [69] |
| 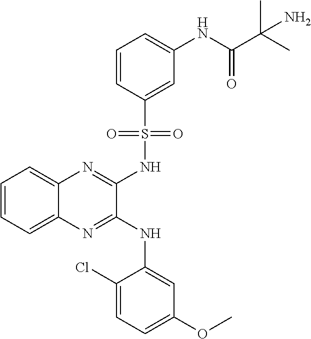 [SMILES: COc1ccc(Cl)c(Nc2nc3ccccc3nc2NS(=O)(=O)c2cccc(NC(=O)C(C)(C)N)c2)c1] | XL-147 Pilaralisib | [70] |
| 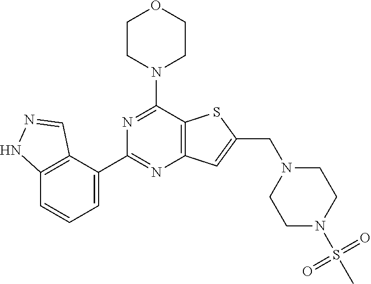 | GDC-0941 (Pictilisib) | [70] |
| 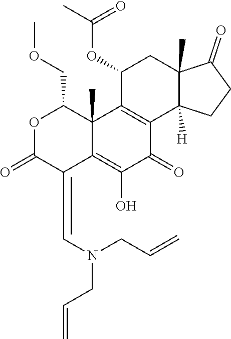 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C] | PX-866 (Sonolisib) | [70] |
| 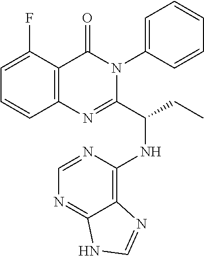 [SMILES: CC[C@H](Nc1ncnc2[nH]cnc12)c1nc2cccc(F)c2c(=O)n1-c1ccccc1] | CAL-101 GS-1101 (Idelalisib) | [70] |

Table 3 is not intended to be a complete list of all possible PI3K/PDK1 Inhibitors that may be of value in this invention. These drugs are an active area of research for cancer and other diseases, and the use of these drugs in the treatment of schizophrenia is a novel utility. Accordingly, as-yet-undiscovered compounds that inhibit PI3K/PDK1 are expected to have anti-schizophrenic activity. The term “small molecule” means that these are drug-like compounds[71] typically with one or more nitrogen-containing heterocycles, a molecular weight typically less than 600, and are not peptides or proteins (but exceptions exist, for example Sonolisib has no nitrogen containing heterocycle but is otherwise a non-peptidyl drug-like compound).

The phenothiazine derivatives listed in Table 3 are methylene blue (MB) and methylene violet (MV) derivatives found to be ferroptosis inhibitors.[67] Ferroptosis is an iron-dependent and ROS-dependent form of cell death accompanied by rapid loss of plasma and mitochondrial membrane integrity. Ferroptosis inhibitors have been investigated for certain neurodegenerative conditions but not schizophrenia (id). The MV derivative with n=6 was the most potent ferroptosis inhibitor (id).

XL-765 induces endoplasmic reticulum (ER) stress dependent apoptosis. The activation of CHOP/DR5 pathway by XL765 induced ER stress is responsible for the induction of apoptosis.[68] Moreover, the inhibition of the mTOR signal by XL-765 is the major source of ER stress, rather than inhibition of PI3K.

### Lipid Replacement Therapy

LRT consists of oral supplementation of membrane glycerophospholipids to provide replacement molecules that are damaged by oxidation in several disorders, including schizophrenia. Glycerophospholipids form the matrix for all cellular membranes and provide separation of enzymatic and chemical reactions into discrete cellular compartments and organelles as well as being essential for the function of membrane enzymes. The lipids are also antioxidant and provide precursors for bioactive molecules that function in signal transduction and molecular recognition pathways. [72]

In embodiment, an LRT composition may be a mixture of at least two components selected from phosphatidylcholine; phosphatidyl-inositol;
phosphatidylethanolamine; phosphatidic acid; and digalactosyl-diacylglycerol. An LRT composition may be a cocktail of lipids and phospholipids that restore cellular membrane integrity.

An exemplary LRT composition is (“NTFactor®”) [72]: phosphatidylcholine (PC) 31.6%;
phosphatidyl-inositol (PI) 24.9%;
phosphatidylethanolamine (PE) 18.9%;
phosphatidic acid 13.9%;
digalactosyl-diacylglycerol 5.9%;
phosphatidylglycerol (PG) 2.4%;
lysophosphatidylcholine 1.0%;
phosphatidyl-serine (PS) 0.5%;
monoglactosyldiacylglycerol 0.3%.
linoleic acid or 18:2D9,12 (n-6) 58.4%;
palmitic acid (16:0) 19.4%;
oleic acid or 18:1 D9 (n-9) 9.7%;
docosahexaenoic acid (DHA) 5.9%;
stearic acid (18:0) 3.9%

First generation antipsychotic drugs are amphiphilic compounds which intercalate themselves into the lipid bilayer, altering the conformation and fluidity of cell membranes [73] FIG. 1). In addition, some of these agents possess antioxidant properties and may reduce lipid peroxidation. [74-75] For example, phenothiazines are PDK1 inhibitors and exhibit antioxidant properties, especially by opposing lipid peroxidation, emphasizing further the antipsychotic properties of the PI3K/PDK1/Akt pathway [76-79]. The Akt pathway inhibits the downstream GSK-3β, while inhibition of Akt reverses GSK-3β suppression. Analysis of GSK-3β phospho-proteome has found that this molecule can be phosphorylated at two sites, serine 9 (ser9) (inhibitory phosphorylation) that reduces the substrate affinity, and tyrosine 216 (Tyr216) (activating phosphorylation) that increases substrate affinity for Akt. [80-81]

The antioxidant properties of LRT and novel phenothiazine derivatives are significant for schizophrenia, a condition characterized not only by increased lipid peroxidation but also by other oxidative markers, including plasma and cerebrospinal fluid (CSF) thiobarbituric acid, exhaled pentane, and urinary isoprostane-8-epi-prostaglandin F2 alpha, [82-85] In addition, antioxidant systems are lowered in schizophrenia, including the enzymes superoxide dismutase (SOD), glutathione peroxidase (GSHPx), and catalase as well as the peripheral non-enzymatic pathways. [86-87] Indeed, phospholipids have been shown to increase SOD and GSHPx by 48% and 48.1% respectively, emphasizing the beneficial role of LRT in schizophrenia. [88]

Several second-generation antipsychotic drugs, including olanzapine and quetiapine, were shown to lower the ER stress, a pathology associated with lipid peroxidation. [89-90] Likewise, OSU-03012 possesses PERK-activating properties that lower ER stress, emphasizing a mechanism of action similar to the second-generation antipsychotic drugs.[91] Moreover, cell membrane lipids are produced in the ER and transported to the plasma membranes, therefore lowering ER stress would improve the quality and turnover of cell membrane lipids. [92-93]

### PI3K/Akt Inhibitors for Schizophrenia

Due to their ability to reverse cellular senescence, PDK1 inhibitors are of extreme interest in schizophrenia, a disorder marked by premature aging, and early mortality. [27, 94, 95] Indeed, several PDK1 inhibitors, including alpha-lipoic acid NAC have been found to lower lipid peroxidation, and are currently being evaluated for schizophrenia [96-100] (NCT03788759).

Akt kinase is activated at the cell membrane by PI3K that in turn activates PDK1, a master kinase, recruited to the plasma membrane via its C-terminal pleckstrin homology (PH) domain. Interestingly, PH polymorphisms were previously associated with schizophrenia, further implicating PDK1 in this disorder. [101]

Overactive Akt/PI3K increases lipid peroxidation which in the presence of iron can trigger ferroptosis. [102] The aging brain retains iron which can enhance oxidation, a pathology associated with schizophrenia, that could be reversed by LRT. [103, 104] Interestingly, aside from attaching to its receptors, DA also binds to the membrane lipids directly, increasing the risk of ferroptosis as it also functions a siderophore that can uptake and release iron, linking this biometal to schizophrenia. [105, 106] In contrast, antipsychotic medications have been associated with iron deficiency anemia, suggesting that these agents promote intracellular iron sequestration, probably explaining the upregulated brain iron. [107, 108] In return, brain iron accumulation upregulates PDK1, emphasizing that drugs such as OSU-03012 may also counteract iron toxicity.[109]

### Combinations

As discussed above, PI3K/PDK1 inhibitors and LRT participate in the same cascade affecting permeability of neuronal bilayer cellular membranes. Thus, combinations of small molecule PI3K/PDK1 inhibitors and LRT expected to be of value in treating schizophrenia and related neurological disorders. Accordingly, in an embodiment, a PDK1 inhibitor is used in combination with LRT as a treatment for schizophrenia.

### Formulations

In an embodiment, a pharmaceutical formulation is provided comprising a small molecule PI3K/PDK1 inhibitor. Such an inhibitor may be a compound selected from Table 3. A formulation may be provided as a liquid or solid dosage form.

For the purposes of the invention, the term “solid dosage form” or “solid composition” refers to dosage forms such as tablets, capsules, or powders that are intended to be swallowed, i.e., used for oral administration. The solid compositions of the present invention can be prepared according to methods well known in the state of the art. Solid compositions typically are formulated with at least one excipient. The appropriate excipients and/or carriers, and their amounts, can readily be determined by those skilled in the art according to the type of formulation being prepared.

The pharmaceutical composition as defined above comprise appropriate excipients or carriers including, but not limited to, binders, fillers, disintegrants, glidants, lubricants or their mixtures. Additionally, the compositions of the present invention may contain other ingredients, such as flavoring agents and colorants, and other components known in the state of the art for use in pharmaceutical compositions.

The term “binder” refers to any pharmaceutically acceptable compound having binding properties. Materials commonly used as binders include povidone such as polyvinylpyrrolidone, methylcellulose polymers, hydroxyethyl cellulose, hydroxypropyl cellulose, L-hydroxypropyl cellulose (low substituted), hydroxypropylmethyl cellulose (HPMC), sodium carboxymethyl cellulose, carboxymethylene, carboxymethylhydroxyethyl cellulose and other cellulose derivatives, starches or modified starches, gelatine, sugars such as sucrose, glucose and sorbitol, gums such as sum arabic, tragacanth, agar and carragenenan; and mixture thereof. In an embodiment, the composition of the invention is one wherein the pharmaceutically or cosmetically acceptable excipients or carriers comprise one or more binder; preferably comprise polyvinylpyrrolidone. In an embodiment, the composition of the invention is one wherein the pharmaceutically or cosmetically acceptable excipients or carriers comprise one or more binder in an amount from 1% to 10% by weight, preferably from 1% to 6% by weight, more preferably from 1% to 3% by weight of the composition.

The terms “filler” and “diluent” have the same meaning and are used interchangeably. They refer to any pharmaceutically acceptable excipient or carrier (material) that fill out the size of a composition, making it practical to produce and convenient for the consumer to use. Materials commonly used as filler include calcium carbonate, calcium phosphate, dibasic calcium phosphate, tribasic calcium sulfate, calcium carboxymethyl cellulose, cellulose, cellulose products such as microcrystalline cellulose and its salts, dextrinderivatives, dextrin, dextrose, fructose, lactitol, lactose, starches or modified starches, magnesium carbonate, magnesium oxide, maltitol, maltodextrins, maltose, mannitol, sorbitol, starch, sucrose, sugar, xylitol, erythritol and mixtures thereof. In an embodiment, the composition of the invention is one wherein the pharmaceutically or cosmetically acceptable excipients or carriers comprises one or more filler; preferably comprises microcrystalline cellulose and its salts.

The term “disintegrant” refers to a substance which helps the composition break up once ingested. Materials commonly used as a disintegrant include cross linked polyvinylpyrolidone; starches such as maize starch and dried sodium starch glycolate; gums such as maize starch and dried sodium starch glycolate; gums such as alginic acid, sodium alginate, guar gum; croscarmellose sodium; low-substituted hydroxypropyl cellulose and mixtures thereof.

The term “glidant” refers to a substance which improves the flow characteristics of powder mixtures in the dry state. Materials commonly used as a glidant include magnesium stearate, colloidal silicon dioxide or talc. In an embodiment, the composition of the invention is one wherein the pharmaceutically or cosmetically acceptable excipients or carriers comprises one or more glidant; preferably comprises magnesium stearate, talc or mixture thereof.

The term “lubricant” refers to a substance that prevents composition ingredients from clumping together and from sticking to the tablet punches or capsule filling machine and improves flowability of the composition mixture. Materials commonly used as a lubricant include sodium oleate, sodium stearate, sodium benzoate, sodium stearate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc and mixtures thereof. The presence of a lubricant is particularly preferred when the composition is a tablet to improve the tableting process. In an embodiment, the composition of the invention is one wherein the pharmaceutically or cosmetically acceptable excipients or carriers comprises one or more lubricants; preferably comprises magnesium stearate.

The pharmaceutical compositions of the present invention can be prepared according to methods well known in the state of the art. The appropriate excipients and/or carriers, and their amounts, can readily be determined by those skilled in the art according to the type of formulation being prepared. In an embodiment, the pharmaceutical composition is a tablet. In an embodiment, the pharmaceutical composition is a direct-compressed tablet. In an embodiment, the pharmaceutical composition is a capsule filled with a powder. In an embodiment, the pharmaceutical composition is a powder intended for suspension or dissolution in a beverage for oral consumption (a drinkable composition).

In an embodiment, the composition may be an injectable dosage form, that can be administered by any injectable route, such as intravenously (IV), intramuscularly (IM), or subcutaneously. Any injectable dosage form needs to be sterile. Injectable dosage forms may be provided as liquids ready for injection, or as a powder or other material intended to be mixed with water or other liquids and prepared just prior to injection.

Examples of suitable aqueous and nonaqueous carriers for injection include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or and antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

To prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Typically, oral dosage forms such as tablets and capsules are used with older children and competent adults who can take instruction and swallow a tablet or capsule. Drinkable dosage forms can be used with small children or incompetent adults who cannot swallow tablets or capsules. Injectable dosage forms may be of value in this invention if a drug is not orally available, or if the patient is unconscious or cannot cooperate with caregivers.

### CONCLUSION

Schizophrenia remains a debilitating, chronic illness in which little progress was made after the discovery of chlorpromazine more than 75 years ago. Moreover, DA antagonists represent symptomatic treatment that does not affect the longitudinal progression of schizophrenia. There is an urgent need for the development of novel etiopathogenetic treatments that could curtail the illness evolution and prevent disability.

Premature brain senescence and oxidation of cell membrane lipids alter both neurotransmission and the integrity of biological barriers that are comprised of cell membranes connected by tight junction molecules.

We propose that LRT and OSU-03012, modalities that inhibit PDK1, represent etiopathogenetic therapies as they correct cell membranes and the biological barriers, averting the CNS entry of toxins and microbes and subsequent pathology. In addition, LRT and small molecule PDK1 inhibitors such as OSU-03012 inhibit overactive Akt and restore microglial homeostasis.

### REFERENCES

[1] T. R. Insel, “Rethinking schizophrenia,” Nature, vol. 468, no. 7321, pp. 187-193, November 2010, doi: 10.1038/nature09552.
[2] C. Constantinides et al., “Brain ageing in schizophrenia: evidence from 26 international cohorts via the ENIGMA Schizophrenia consortium,” Mol Psychiatry, vol. 28, no. 3, pp. 1201-1209, March 2023, doi: 10.1038/s41380-022-01897-w.
[3] N. Koutsouleris et al., “Accelerated Brain Aging in Schizophrenia and Beyond: A Neuroanatomical Marker of Psychiatric Disorders,” Schizophr Bull, vol. 40, no. 5, pp. 1140-1153, September 2014, doi: 10.1093/schbul/sbtl42.
[4] U. N. Das, “‘Cell Membrane Theory of Senescence’ and the Role of Bioactive Lipids in Aging, and Aging Associated Diseases and Their Therapeutic Implications,” Biomolecules, vol. 11, no. 2, p. 241, February 2021, doi: 10.3390/biom11020241.
[5] T. Kaufmann et al., “Common brain disorders are associated with heritable patterns of apparent aging of the brain,” Nat Neurosci, vol. 22, no. 10, pp. 1617-1623, October 2019, doi: 10.1038/s41593-019-0471-7.
[6] M. Li et al., “Impaired Membrane Lipid Homeostasis in Schizophrenia,” Schizophr Bull, vol. 48, no. 5, pp. 1125-1135, September 2022, doi: 10.1093/schbul/sbac011.
[7] S. Hamsanathan and A. U. Gurkar, “Lipids as Regulators of Cellular Senescence,” Front Physiol, vol. 13, March 2022, doi: 10.3389/fphys.2022.796850.
[8] M. Jove, I. Pradas, M. Dominguez-Gonzalez, I. Ferrer, and R. Pamplona, “Lipids and lipoxidation in human brain aging. Mitochondrial ATP-synthase as a key lipoxidation target,” Redox Biol, vol. 23, p. 101082, May 2019, doi: 10.1016/j.redox.2018.101082.
[9] A. Elbaradei, Z. Wang, and N. Malmstadt, “Oxidation of Membrane Lipids Alters the Activity of the Human Serotonin 1A Receptor,” Langmuir, vol. 38, no. 22, pp. 6798-6807, June 2022, doi: 10.1021/acs.langmuir.1c03238.
[10] A. Sfera et al., “Receptor-Independent Therapies for Forensic Detainees with Schizophrenia-Dementia Comorbidity,” Int J Mol Sci, vol. 24, no. 21, p. 15797, October 2023, doi: 10.3390/ijms242115797.
[11] W. Zheng et al., “The possible role of the Akt signaling pathway in schizophrenia,” Brain Res, vol. 1470, pp. 145-158, August 2012, doi: 10.1016/j.brainres.2012.06.032.
[12] K. T. Chan et al., “A functional genetic screen defines the AKT-induced senescence signaling network,” Cell Death Differ, vol. 27, no. 2, pp. 725-741, February 2020, doi: 10.1038/s41418-019-0384-8.
[13] M.-L. Chen et al., “Antipsychotic drugs suppress the AKT/NF-κB pathway and regulate the differentiation of T-cell subsets,” Immunol Lett, vol. 140, no. 1-2, pp. 81-91, October 2011, doi: 10.1016/j.imlet.2011.06.011.
[14] E. S. Emamian, D. Hall, M. J. Birnbaum, M. Karayiorgou, and J. A. Gogos, “Convergent evidence for impaired AKT1-GSK33 signaling in schizophrenia,” Nat Genet, vol. 36, no. 2, pp. 131-137, February 2004, doi: 10.1038/ng1296.
[15] K. Brami-Cherrier, E. Valjent, M. Garcia, C. Pages, R. A. Hipskind, and J. Caboche, “Dopamine Induces a PI3-Kinase-Independent Activation of Akt in Striatal Neurons: A New Route to cAMP Response Element-Binding Protein Phosphorylation,” The Journal of Neuroscience, vol. 22, no. 20, pp. 8911-8921, October 2002, doi: 10.1523/JNEUROSCI.22-20-08911.2002.
[16] J. Sánchez-Wandelmer et al., “Effects of the antipsychotic drug haloperidol on the somastostatinergic system in SH-SY5Y neuroblastoma cells,” J Neurochem, vol. 110, no. 2, pp. 631-640, July 2009, doi: 10.1111/j.1471-4159.2009.06159.x.
[17] S. Zhang et al., “OSU-03012, a Novel Celecoxib Derivative, Is Cytotoxic to Myeloma Cells and Acts through Multiple Mechanisms,” Clinical Cancer Research, vol. 13, no. 16, pp. 4750-4758, August 2007, doi: 10.1158/1078-0432.CCR-07-0136.
[18] J. Mateo et al., “A first-in-human phase I trial of AR-12, a PDK-1 inhibitor, in patients with advanced solid tumors.,” Journal of Clinical Oncology, vol. 31, no. 15_suppl, pp. 2608-2608, May 2013, doi: 10.1200/jco.2013.31.15_suppl.2608.
[19] S. N. Rai et al., “The Role of PI3K/Akt and ERK in Neurodegenerative Disorders,” Neurotox Res, vol. 35, no. 3, pp. 775-795, April 2019, doi: 10.1007/s12640-019-0003-y.
[20] A. Cianciulli, C. Porro, R. Calvello, T. Trotta, D. D. Lofrumento, and M. A. Panaro, “Microglia Mediated Neuroinflammation: Focus on PI3K Modulation,” Biomolecules, vol. 10, no. 1, p. 137, January 2020, doi: 10.3390/biom10010137.
[21] P. Katsel et al., “Abnormal Indices of Cell Cycle Activity in Schizophrenia and their Potential Association with Oligodendrocytes,” Neuropsychopharmacology, vol. 33, no. 12, pp. 2993-3009, November 2008, doi: 10.1038/npp.2008.19.
[22] S. Okazaki et al., “The cell cycle-related genes as biomarkers for schizophrenia,” Prog Neuropsychopharmacol Biol Psychiatry, vol. 70, pp. 85-91, October 2016, doi: 10.1016/j.pnpbp.2016.05.005.
[23] K. Bhaskar, M. Miller, A. Chludzinski, K. Herrup, M. Zagorski, and B. T. Lamb, “The PI3K-Akt-mTOR pathway regulates Abeta oligomer induced neuronal cell cycle events.,” Mol Neurodegener, vol. 4, p. 14, March 2009, doi: 10.1186/1750-1326-4-14.
[24] S. Nandakumar, E. Rozich, and L. Buttitta, “Cell Cycle Re-entry in the Nervous System: From Polyploidy to Neurodegeneration,” Front Cell Dev Biol, vol. 9, June 2021, doi: 10.3389/fcell.2021.698661.
[25] N. Gao et al., “G₁ cell cycle progression and the expression of G 1 cyclins are regulated by PI3K/AKT/mTOR/p70S6K1 signaling in human ovarian cancer cells,” American Journal of Physiology-Cell Physiology, vol. 287, no. 2, pp. C281-C291, August 2004, doi: 10.1152/ajpcell.00422.2003.
[26] J. Liang and J. M. Slingerland, “Multiple roles of the PI3K/PKB (Akt) pathway in cell cycle progression.,” Cell Cycle, vol. 2, no. 4, pp. 339-45, 2003.
[27] S. An et al., “Inhibition of 3-phosphoinositide-dependent protein kinase 1 (PDK1) can revert cellular senescence in human dermal fibroblasts,” Proceedings of the National Academy of Sciences, vol. 117, no. 49, pp. 31535-31546, December 2020, doi: 10.1073/pnas.1920338117.
[28] S. Yang, Y. Du, X. Zhao, C. Wu, and P. Yu, “Reducing PDK1/Akt Activity: An Effective Therapeutic Target in the Treatment of Alzheimer's Disease,” Cells, vol. 11, no. 11, p. 1735, May 2022, doi: 10.3390/cells11111735.
[29] H. Kim, J. Lee, and Y. Cho, “PDK1 is a negative regulator of axon regeneration.,” Mol Brain, vol. 14, no. 1, p. 31, February 2021, doi: 10.1186/s13041-021-00748-z.
[30] L. E. Laskaris et al., “Microglial activation and progressive brain changes in schizophrenia,” Br J Pharmacol, vol. 173, no. 4, pp. 666-680, February 2016, doi: 10.1111/bph.13364.
[31] D. Kong and T. Yamori, “ZSTK474, a novel phosphatidylinositol 3-kinase inhibitor identified using the JFCR39 drug discovery system,” Acta Pharmacol Sin, vol. 31, no. 9, pp. 1189-1197, September 2010, doi: 10.1038/aps.2010.150.
[32] P. Wang et al., “Class I PI3K inhibitor ZSTK474 mediates a shift in microglial/macrophage phenotype and inhibits inflammatory response in mice with cerebral ischemia/reperfusion injury,” J Neuroinflammation, vol. 13, no. 1, p. 192, December 2016, doi: 10.1186/si2974-016-0660-1.
[33] F. Lin et al., “PI3K-mTOR Pathway Inhibition Exhibits Efficacy Against High-grade Glioma in Clinically Relevant Mouse Models,” Clinical Cancer Research, vol. 23, no. 5, pp. 1286-1298, March 2017, doi: 10.1158/1078-0432.CCR-16-1276.
[34] X. Han, “Neurolipidomics: challenges and developments,” Frontiers in Bioscience, vol. 12, no. 1, p. 2601, 2007, doi: 10.2741/2258.
[35] J. K. Yao, J. A. Stanley, R. D. Reddy, M. S. Keshavan, and J. W. Pettegrew, “Correlations between peripheral polyunsaturated fatty acid content and in vivo membrane phospholipid metabolites,” Biol Psychiatry, vol. 52, no. 8, pp. 823-830, October 2002, doi: 10.1016/S0006-3223(02)01397-5.
[36] W. P. Hoen, J. G. Lijmer, M. Duran, R. J. A. Wanders, N. J. M. van Beveren, and L. de Haan, “Red blood cell polyunsaturated fatty acids measured in red blood cells and schizophrenia: A meta-analysis,” Psychiatry Res, vol. 207, no. 1-2, pp. 1-12, May 2013, doi: 10.1016/j.psychres.2012.09.041.
[37] P. Nuss et al., “Abnormal transbilayer distribution of phospholipids in red blood cell membranes in schizophrenia,” Psychiatry Res, vol. 169, no. 2, pp. 91-96, September 2009, doi: 10.1016/j.psychres.2009.01.009.
[38] S. Smesny et al., “Skin Ceramide Alterations in First-Episode Schizophrenia Indicate Abnormal Sphingolipid Metabolism,” Schizophr Bull, vol. 39, no. 4, pp. 933-941, July 2013, doi: 10.1093/schbul/sbs058.
[39] A. Frajerman et al., “Lipides membranaires dans la schizophrénie et la psychose débutante: de potentiels biomarqueurs et pistes thérapeutiques?,” Encephale, vol. 46, no. 3, pp. 209-216, June 2020, doi: 10.1016/j.encep.2019.11.009.
[40] Q. Leyrolle, S. Layé, and A. Nadjar, “Direct and indirect effects of lipids on microglia function,” Neurosci Lett, vol. 708, p. 134348, August 2019, doi: 10.1016/j.neulet.2019.134348.
[41] K. Charriere, I. Ghzaiel, G. Lizard, and A. Vejux, “Involvement of Microglia in Neurodegenerative Diseases: Beneficial Effects of Docosahexahenoic Acid (DHA) Supplied by Food or Combined with Nanoparticles,” Int J Mol Sci, vol. 22, no. 19, p. 10639, September 2021, doi: 10.3390/ijms221910639.
[42] V. De Smedt-Peyrusse, F. Sargueil, A. Moranis, H. Harizi, S. Mongrand, and S. Layé, “Docosahexaenoic acid prevents lipopolysaccharide-induced cytokine production in microglial cells by inhibiting lipopolysaccharide receptor presentation but not its membrane subdomain localization,” J Neurochem, vol. 105, no. 2, pp. 296-307, April 2008, doi: 10.1111/j.1471-4159.2007.05129.x.
[43] J. H. Yoon et al., “Brain lipidomics: From functional landscape to clinical significance,” Sci Adv, vol. 8, no. 37, September 2022, doi: 10.1126/sciadv.adc9317.
[44] Z. Gu et al., “Polyunsaturated fatty acids affect the localization and signaling of PIP3/AKT in prostate cancer cells,” Carcinogenesis, vol. 34, no. 9, pp. 1968-1975, September 2013, doi: 10.1093/carcin/bgt147.
[45] X. Li and R. S. Jope, “Is Glycogen Synthase Kinase-3 a Central Modulator in Mood Regulation?,” Neuropsychopharmacology, vol. 35, no. 11, pp. 2143-2154, October 2010, doi: 10.1038/npp.2010.105.
[46] S. W. Park et al., “Effects of antipsychotic drugs on BDNF, GSK-3β, and μ3-catenin expression in rats subjected to immobilization stress,” Neurosci Res, vol. 71, no. 4, pp. 335-340, December 2011, doi: 10.1016/j.neures.2011.08.010.
[47] K. Brami-Cherrier et al., “Parsing Molecular and Behavioral Effects of Cocaine in Mitogen- and Stress-Activated Protein Kinase-1-Deficient Mice,” The Journal of Neuroscience, vol. 25, no. 49, pp. 11444-11454, December 2005, doi: 10.1523/JNEUROSCI.1711-05.2005.
[48] F. Checler, “Alzheimer's and prion diseases: PDK1 at the crossroads,” Nat Med, vol. 19, no. 9, pp. 1088-1090, September 2013, doi: 10.1038/nm.3332.
[49] L. L. O. Sanders et al., “α-Lipoic Acid as Adjunctive Treatment for Schizophrenia,” J Clin Psychopharmacol, vol. 37, no. 6, pp. 697-701, December 2017, doi: 10.1097/JCP.0000000000000800.
[50] E. Neill et al., “N-Acetylcysteine (NAC) in Schizophrenia Resistant to Clozapine: A Double-Blind, Randomized, Placebo-Controlled Trial Targeting Negative Symptoms,” Schizophr Bull, vol. 48, no. 6, pp. 1263-1272, November 2022, doi: 10.1093/schbul/sbac065.
[51] K. H. Tachiki, T. D. Buckman, S. Eiduson, A. S. Kling, and J. Hullett, “Phosphatidylserine inhibition of monoamine oxidase in platelets of schizophrenics,” Biol Psychiatry, vol. 21, no. 1, pp. 59-68, January 1986, doi: 10.1016/0006-3223(86)90008-9.
[52] Y.-L. Wei, C.-X. Li, S.-B. Li, Y. Liu, and L. Hu, “Association study of monoamine oxidase A/B genes and schizophrenia in Han Chinese,” Behavioral and Brain Functions, vol. 7, no. 1, p. 42, December 2011, doi: 10.1186/1744-9081-7-42.
[53] C. Venkataramaiah, S. Payani, B. L. Priya, and J. A. Pradeepkiran, “Therapeutic potentiality of a new flavonoid against ketamine induced glutamatergic dysregulation in schizophrenia: In vivo and in silico approach,” Biomedicine & Pharmacotherapy, vol. 138, p. 111453, June 2021, doi: 10.1016/j.biopha.2021.111453.
[54] B. ul Islam et al., “Flavonoids and PI3K/Akt/mTOR Signaling Cascade: A Potential Crosstalk in Anticancer Treatment,” Curr Med Chem, vol. 28, no. 39, pp. 8083-8097, December 2021, doi: 10.2174/0929867328666210804091548.
[55] H.-W. Zhang et al., “Flavonoids inhibit cell proliferation and induce apoptosis and autophagy through downregulation of PI3Kγ mediated PI3K/AKT/mTOR/p70S6K/ULK signaling pathway in human breast cancer cells,” Sci Rep, vol. 8, no. 1, p. 11255, July 2018, doi: 10.1038/s41598-018-29308-7.
[56] N. Kozlovsky, W. T. Regenold, J. Levine, A. Rapoport, R. H. Belmaker, and G. Agam, “GSK-3? in cerebrospinal fluid of schizophrenia patients,” J Neural Transm, vol. 111, no. 8, August 2004, doi: 10.1007/s00702-003-0127-0.
[57] J. O. Rayner et al., “AR12 (OSU-03012) suppresses GRP78 expression and inhibits SARS-CoV-2 replication,” Biochem Pharmacol, vol. 182, p. 114227, December 2020, doi: 10.1016/j.bcp.2020.114227.
[58] N. Tsao et al., “AR-12 Has a Bactericidal Activity and a Synergistic Effect with Gentamicin against Group A Streptococcus,” Int J Mol Sci, vol. 22, no. 21, p. 11617, October 2021, doi: 10.3390/ijms222111617.
[59] H. Nehme et al., “Antibacterial activity of antipsychotic agents, their association with lipid nanocapsules and its impact on the properties of the nanocarriers and on antibacterial activity,” PLoS One, vol. 13, no. 1, p. e0189950, January 2018, doi: 10.1371/journal.pone.0189950.
[60] S. Matteoni et al., “Anticancer Properties of the Antipsychotic Drug Chlorpromazine and Its Synergism With Temozolomide in Restraining Human Glioblastoma Proliferation In Vitro,” Front Oncol, vol. 11, February 2021, doi: 10.3389/fonc.2021.635472.
[61] R. R. Girgis and J. A. Lieberman, “Anti-viral properties of antipsychotic medications in the time of COVID-19,” Psychiatry Res, vol. 295, p. 113626, January 2021, doi: 10.1016/j.psychres.2020.113626.
[62] L. Tao et al., “Lysosomal membrane permeabilization mediated apoptosis involve in perphenazine-induced hepatotoxicity in vitro and in vivo,” Toxicol Lett, vol. 367, pp. 76-87, August 2022, doi: 10.1016/j.toxlet.2022.07.814.
[63] A. Yacoub et al., “OSU-03012 Promotes Caspase-Independent but PERK-, Cathepsin B-, BID-, and AIF-Dependent Killing of Transformed Cells,” Mol Pharmacol, vol. 70, no. 2, pp. 589-603, August 2006, doi: 10.1124/mol.106.025007.
[64] M. Varalda et al., “Psychotropic Drugs Show Anticancer Activity by Disrupting Mitochondrial and Lysosomal Function,” Front Oncol, vol. 10, October 2020, doi: 10.3389/fonc.2020.562196.
[65] G. D. Smedley, K. E. Walker, and S. H. Yuan, “The Role of PERK in Understanding Development of Neurodegenerative Diseases,” Int J Mol Sci, vol. 22, no. 15, p. 8146, July 2021, doi: 10.3390/ijms22158146.
[66] R. G. Keynes et al., “N¹⁰-carbonyl-substituted phenothiazines inhibiting lipid peroxidation and associated nitric oxide consumption powerfully protect brain tissue against oxidative stress,” Chem Biol Drug Des, vol. 94, no. 3, pp. 1680-1693, September 2019, doi: 10.1111/cbdd.13572.
[67] J. Liu, I. Bandyopadhyay, L. Zheng, O. M. Khdour, and S. M. Hecht, “Antiferroptotic Activity of Phenothiazine Analogues: A Novel Therapeutic Strategy for Oxidative Stress Related Disease,” ACS Med Chem Lett, vol. 11, no. 11, pp. 2165-2173, November 2020, doi: 10.1021/acsmedchemlett.0c00293.
[68] H. Zhao, G. Chen, and H. Liang, “<p>Dual PI3K/mTOR Inhibitor, XL765, suppresses glioblastoma growth by inducing ER stress-dependent apoptosis</p>,” Onco Targets Ther, vol. Volume 12, pp. 5415-5424, July 2019, doi: 10.2147/OTT.S210128.
[69] S. Duggan and Z. T. Al-Salama, “Leniolisib: First Approval,” Drugs, vol. 83, no. 10, pp. 943-948, July 2023, doi: 10.1007/s40265-023-01895-4.
[70] P. Wu and Y.-Z. Hu, “PI3K/Akt/mTOR Pathway Inhibitors in Cancer: A Perspective on Clinical Progress,” Curr Med Chem, vol. 17, no. 35, pp. 4326-4341, December 2010, doi: 10.2174/092986710793361234.
[71] C. A. Lipinski, F. Lombardo, B. W. Dominy, and P. J. Feeney, “Experimental and computational approaches to estimate solubility and permeability in drug discovery and development settings 1PII of original article: S0169-409X(96)00423-1. The article was originally published in Advanced Drug Delivery Reviews 23 (1997) 3-25. 1,” Adv Drug Deliv Rev, vol. 46, no. 1-3, pp. 3-26, March 2001, doi: 10.1016/SO169-409X(00)00129-0.
[72] G. L. Nicolson et al., “Clinical Uses of Membrane Lipid Replacement Supplements in Restoring Membrane Function and Reducing Fatigue,” discoveries, vol. 4, no. 1, p. e54, February 2016, doi: 10.15190/d.2016.1.
[73] R. Oruch, A. Lund, I. F. Pryme, and H. Holmsen, “An intercalation mechanism as a mode of action exerted by psychotropic drugs: results of altered phospholipid substrate availabilities in membranes?,” J Chem Biol, vol. 3, no. 2, pp. 67-88, May 2010, doi: 10.1007/si2154-009-0034-6.
[74] M. Dalla Tiezza, T. A. Hamlin, F. M. Bickelhaupt, and L. Orian, “Radical Scavenging Potential of the Phenothiazine Scaffold: A Computational Analysis,” ChemMedChem, vol. 16, no. 24, pp. 3763-3771, December 2021, doi: 10.1002/cmdc.202100546.
[75] A. N. Matralis and A. P. Kourounakis, “Design of Novel Potent Antihyperlipidemic Agents with Antioxidant/Anti-inflammatory Properties: Exploiting Phenothiazine's Strong Antioxidant Activity,” J Med Chem, vol. 57, no. 6, pp. 2568-2581, March 2014, doi: 10.1021/jm401842e.
[76] O. Voronova, S. Zhuravkov, E. Korotkova, A. Artamonov, and E. Plotnikov, “Antioxidant Properties of New Phenothiazine Derivatives,” Antioxidants, vol. 11, no. 7, p. 1371, July 2022, doi: 10.3390/antiox11071371.
[77] J. H. Choi et al., “Potential Inhibition of PDK1/Akt Signaling by Phenothiazines Suppresses Cancer Cell Proliferation and Survival,” Ann N Y Acad Sci, vol. 1138, no. 1, pp. 393-403, September 2008, doi: 10.1196/annals.1414.041.
[78] D. Roy, D. N. Pathak, and R. Singh, “Effects of Chlorpromazine on the Activities of Antioxidant Enzymes and Lipid Peroxidation in the Various Regions of Aging Rat Brain,” J Neurochem, vol. 42, no. 3, pp. 628-633, March 1984, doi: 10.1111/j.1471-4159.1984.tb02728.x.
[79] M. J. Yu et al., “Phenothiazines as lipid peroxidation inhibitors and cytoprotective agents,” J Med Chem, vol. 35, no. 4, pp. 716-724, February 1992, doi: 10.1021/jm00082a012.
[80] B. Welz et al., “Activation of GSK3 Prevents Termination of TNF-Induced Signaling,” J Inflamm Res, vol. Volume 14, pp. 1717-1730, May 2021, doi: 10.2147/JIR. S300806.
[81] E. ter Haar, J. T. Coll, D. A. Austen, H.-M. Hsiao, L. Swenson, and J. Jain, “Structure of GSK33 reveals a primed phosphorylation mechanism,” Nat Struct Biol, vol. 8, no. 7, pp. 593-596, 2001, doi: 10.1038/89624.
[82] M. Phillips, M. Sabas, and J. Greenberg, “Increased pentane and carbon disulfide in the breath of patients with schizophrenia.,” J Clin Pathol, vol. 46, no. 9, pp. 861-4, September 1993, doi: 10.1136/jcp.46.9.861.
[83] B. K. Y. Bitanihirwe and T.-U. W. Woo, “Oxidative stress in schizophrenia: An integrated approach,” Neurosci Biobehav Rev, vol. 35, no. 3, pp. 878-893, January 2011, doi: 10.1016/j.neubiorev.2010.10.008.
[84] A. Dietrich-Muszalska and B. Olas, “Isoprostenes as indicators of oxidative stress in schizophrenia,” The World Journal of Biological Psychiatry, vol. 10, no. 1, pp. 27-33, January 2009, doi: 10.1080/15622970701361263.
[85] S. P. Mahadik and D. R. Evans, “Is schizophrenia a metabolic brain disorder?Membrane phospholipid dysregulation and its therapeutic implications,” Psychiatric Clinics of North America, vol. 26, no. 1, pp. 85-102, March 2003, doi: 10.1016/SO193-953X(02)00033-3.
[86] Z. Lu et al., “Oxidative Stress and Psychiatric Disorders: Evidence from the Bidirectional Mendelian Randomization Study,” Antioxidants, vol. 11, no. 7, p. 1386, July 2022, doi: 10.3390/antioxl1071386.
[87] G. Dadheech, S. Mishra, S. Gautam, and P. Sharma, “Evaluation of antioxidant deficit in schizophrenia,” Indian J Psychiatry, vol. 50, no. 1, p. 16, 2008, doi: 10.4103/0019-5545.39753.
[88] L. ABENAVOLI et al., “Treatment with phosphatidylcholine of patients with nonalcoholic fatty liver disease: a prospective pilot study,” Minerva Gastroenterology, vol. 68, no. 4, December 2022, doi: 10.23736/S2724-5985.21.03066-7.
[89] S. Kurosawa, E. Hashimoto, W. Ukai, S. Toki, S. Saito, and T. Saito, “Olanzapine potentiates neuronal survival and neural stem cell differentiation: regulation of endoplasmic reticulum stress response proteins,” J Neural Transm, vol. 114, no. 9, pp. 1121-1128, September 2007, doi: 10.1007/s00702-007-0747-z.
[90] D. Grajales, P. Vázquez, R. Alén, A. B. Hitos, and Á. M. Valverde, “Attenuation of Olanzapine-Induced Endoplasmic Reticulum Stress Improves Insulin Secretion in Pancreatic Beta Cells,” Metabolites, vol. 12, no. 5, p. 443, May 2022, doi: 10.3390/metabo12050443.
[91] L. Booth et al., “OSU-03012 suppresses GRP78/BiP expression that causes PERK-dependent increases in tumor cell killing,” Cancer Biol Ther, vol. 13, no. 4, pp. 224-236, February 2012, doi: 10.4161/cbt.13.4.18877.
[92] J. Jacquemyn, A. Cascalho, and R. E. Goodchild, “The ins and outs of endoplasmic reticulum-controlled lipid biosynthesis,” EMBO Rep, vol. 18, no. 11, pp. 1905-1921, November 2017, doi: 10.15252/embr.201643426.
[93] P. Fagone and S. Jackowski, “Membrane phospholipid synthesis and endoplasmic reticulum function,” J Lipid Res, vol. 50, pp. S311-S316, April 2009, doi: 10.1194/jlr.R800049-JLR200.
[94] D. Paramos-de-Carvalho, A. Jacinto, and L. Saúde, “The right time for senescence,” Elife, vol. 10, November 2021, doi: 10.7554/eLife.72449.
[95] L. Zhang, L. E. Pitcher, V. Prahalad, L. J. Niedernhofer, and P. D. Robbins, “Targeting cellular senescence with senotherapeutics: senolytics and senomorphics,” FEBS J, vol. 290, no. 5, pp. 1362-1383, March 2023, doi: 10.1111/febs.16350.
[96] L. Yue et al., “α-Lipoic Acid Targeting PDK1/NRF2 Axis Contributes to the Apoptosis Effect of Lung Cancer Cells,” Oxid Med Cell Longev, vol. 2021, pp. 1-16, June 2021, doi: 10.1155/2021/6633419.
[97] S. S. Hann, F. Zheng, and S. Zhao, “Targeting 3-phosphoinositide-dependent protein kinase 1 by N-acetyl-cysteine through activation of peroxisome proliferators activated receptor alpha in human lung cancer cells, the role of p53 and p65,” Journal of Experimental & Clinical Cancer Research, vol. 32, no. 1, p. 43, December 2013, doi: 10.1186/1756-9966-32-43.
[98] S. L. Rossell et al., “N-acetylcysteine (NAC) in schizophrenia resistant to clozapine: a double blind randomised placebo controlled trial targeting negative symptoms,” BMC Psychiatry, vol. 16, no. 1, p. 320, December 2016, doi: 10.1186/s12888-016-1030-3.
[99] L. L. O. Sanders et al., “α-Lipoic Acid as Adjunctive Treatment for Schizophrenia,” J Clin Psychopharmacol, vol. 37, no. 6, pp. 697-701, December 2017, doi: 10.1097/JCP.0000000000000800.

A. Mishra, K. H. Reeta, S. C. Sarangi, R. Maiti, and M. Sood, “Effect of add-on alpha lipoic acid on psychopathology in patients with treatment-resistant schizophrenia: a pilot randomized double-blind placebo-controlled trial,” Psychopharmacology (Berl), vol. 239, no. 11, pp. 3525-3535, November 2022, doi: 10.1007/s00213-022-06225-2.
[101] I. Spellmann et al., “Pleckstrin homology domain containing 6 protein (PLEKHA6) polymorphisms are associated with psychopathology and response to treatment in schizophrenic patients,” Prog Neuropsychopharmacol Biol Psychiatry, vol. 51, pp. 190-195, June 2014, doi: 10.1016/j.pnpbp.2014.02.006.
[102] J. Yi, J. Zhu, J. Wu, C. B. Thompson, and X. Jiang, “Oncogenic activation of PI3K-AKT-mTOR signaling suppresses ferroptosis via SREBP-mediated lipogenesis,” Proceedings of the National Academy of Sciences, vol. 117, no. 49, pp. 31189-31197, December 2020, doi: 10.1073/pnas.2017152117.
[103] S. Feng et al., “Identification of Ferroptosis-Related Genes in Schizophrenia Based on Bioinformatic Analysis,” Genes (Basel), vol. 13, no. 11, p. 2168, November 2022, doi: 10.3390/genes13112168.
[104] T. Sato, J. S. Shapiro, H.-C. Chang, R. A. Miller, and H. Ardehali, “Aging is associated with increased brain iron through cortex-derived hepcidin expression,” Elife, vol. 11, January 2022, doi: 10.7554/eLife.73456.
[105] F. Lolicato et al., “Membrane-Dependent Binding and Entry Mechanism of Dopamine into Its Receptor,” ACS Chem Neurosci, vol. 11, no. 13, pp. 1914-1924, July 2020, doi: 10.1021/acschemneuro.9b00656.
[106] S. Dichtl et al., “Dopamine Is a Siderophore-Like Iron Chelator That Promotes Salmonella enterica Serovar Typhimurium Virulence in Mice,” mBio, vol. 10, no. 1, February 2019, doi: 10.1128/mBio.02624-18.
[107] A. Z. Wasti, S. Zahid, and N. Ahmed, “Antipsychotic Drugs Induced Iron Deficiency Anemia in Schizophrenic Patients,” 2013. [Online]. Available: http://www.journalijar.com
[108] A. Lotan et al., “Perturbed iron biology in the prefrontal cortex of people with schizophrenia,” Mol Psychiatry, vol. 28, no. 5, pp. 2058-2070, May 2023, doi: 10.1038/s41380-023-01979-3.
[109] K. Chen et al., “Loss of Frataxin induces iron toxicity, sphingolipid synthesis, and Pdk1/Mef2 activation, leading to neurodegeneration,” Elife, vol. 5, June 2016, doi: 10.7554/eLife.16043.

1. The use of a PI3K/PDK1 inhibitor for the treatment of schizophrenia.

2. The use of a PI3K/PDK1 inhibitor according to claim 1, wherein the PI3K/PDK1 inhibitor is a compound from selected from:

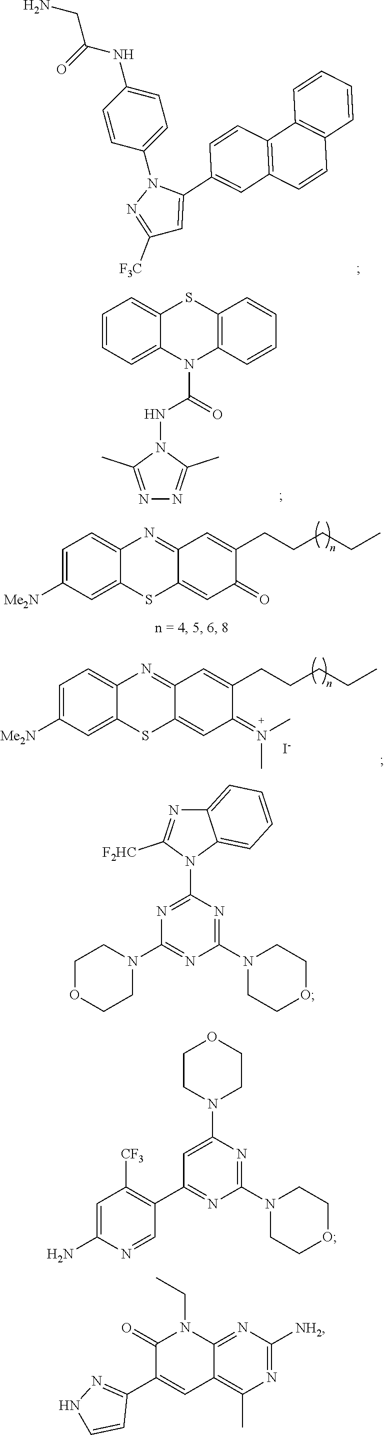

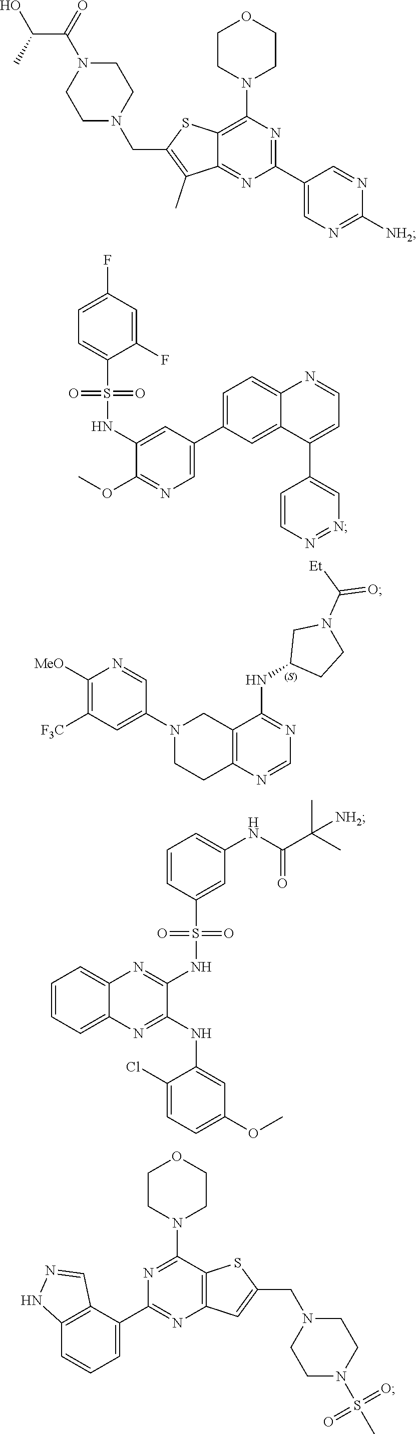

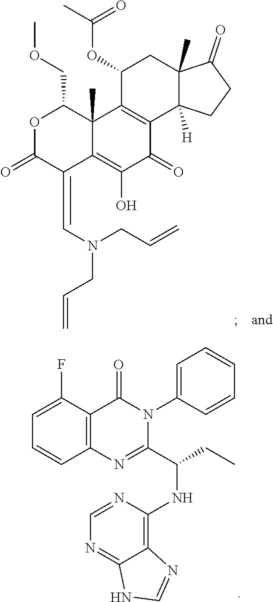

3. A method of treating or preventing the onset of schizophrenia comprising the administration of lipid replacement therapy comprising a mixture of at least two components selected from phosphatidylcholine; phosphatidyl-inositol; phosphatidylethanolamine; phosphatidic acid; and digalactosyl-diacylglycerol.

4. The method of claim 3 comprising a cocktail consisting of phosphatidylcholine (PC) 31.6%; phosphatidyl-inositol (PI) 24.9%; phosphatidylethanolamine (PE) 18.9%; phosphatidic acid 13.9%; digalactosyl-diacylglycerol 5.9%; phosphatidylglycerol (PG) 2.4%; lysophosphatidylcholine 1.0%; phosphatidyl-serine (PS) 0.5%; monoglactosyldiacylglycerol 0.3%; linoleic acid or 18:2D9,12 (n-6) 58.4%; palmitic acid (16:0) 19.4%; oleic acid or 18:1D9 (n-9) 9.7%; docosahexaenoic acid (DHA) 5.9%; and stearic acid (18:0) 3.9%.

5. A method of treating or preventing the onset of schizophrenia comprising a combination of a PI3K/PDK1 inhibitor and a lipid replacement therapy comprising a mixture of at least two components selected from phosphatidylcholine; phosphatidyl-inositol; phosphatidylethanolamine; phosphatidic acid; and digalactosyl-diacylglycerol.
